(19) United States
(12) Patent Application Publication
(10) Pub. No.: US 2011/0123576 A1
(43) Pub. Date: May 26, 2011

(54) INTESTINAL ENVIRONMENT-IMPROVING AGENT

(76) Inventors: Noriyuki Suzuki, Tokyo (JP); Kyoko Morishita, Tokyo (JP); Michiaki Murakoshi, Tokyo (JP)

(21) Appl. No.: 13/003,254

(22) PCT Filed: Jul. 9, 2009
(86) PCT No.: PCT/JP2009/062509

§ 371 Date: Jan. 7, 2011

(30) Foreign Application Priority Data
Jul. 10, 2008 (JP) 2008-179710

(51) Int. Cl. A61K 35/74; A61K 9/00; A61P 1/14; A61P 1/00; A61P 35/00

(52) U.S. Cl. 424/400; 424/93.45; 424/780

(57) ABSTRACT

Disclosed is an intestinal environment-improving agent including a lactoferrin and bacteria of Lactobacillus brevis as effective ingredients.

### TECHNICAL FIELD

This invention relates to an intestinal environment-improving agent having an excellent intestinal environment-improving effect.

### BACKGROUND ART

Because of insufficient ingestion of food and food fibers, hormone imbalances and the spiritual stress based on worries and surroundings, there may occur symptoms such as a degraded intestinal environment, and a reduced defecation frequency and stool output. This degradation of the intestinal environment may lead to concern that it involves one of causes for large bowel cancer, intestinal obstruction and the like. On the other hand, although it has been confirmed that when ingesting bacteria of Lactobacillus brevis, immunostimulatory activity and intestinal age score are improved, there is a further demand of one that is in safety and excellent in intestinal environment-improving effect.

### PRIOR-ART DOCUMENTS

### Patent Documents

Patent Document 1: JP-A 2005-068060
Patent Document 2: JP-A 2007-084533

### DISCLOSURE OF THE INVENTION

### Problems to be Solved by the Invention

The invention has been made under such circumstances as set out above. And an object of the invention is to provide an intestinal environment-improving agent having an excellent intestinal environment-improving effect.

### Means for Solving the Problems

We have made intensive studies in order to achieve the above object and, as a result, found that when lactoferrin and bacteria of Lactobacillus brevis are used in combination, an intestinal environment is improved to obtain an appropriate defecation frequency and stool output, thereby arriving at completion of the invention.

Accordingly, the invention provides following intestinal environment-improving agent.

[1] An intestinal environment-improving agent comprising a lactoferrin and bacteria of Lactobacillus brevis as effective ingredients.
[2] The intestinal environment-improving agent as recited in [1], wherein the agent is an enteric coated preparation.
[3] The intestinal environment-improving agent as recited in [1] or [2], wherein the agent comprises not less than one hundred millions of viable bacteria of Lactobacillus brevis.
[4] The intestinal environment-improving agent as recited in [1] or [2], wherein the agent comprises not less than one billion of killed bacteria of Lactobacillus brevis.
[5] The intestinal environment-improving agent as recited in any one of [1] to [4], further comprising an extract of a plant selected from piperaceous, zingiberaceous and solanaceous plants.

### Advantageous Effect of the Invention

According to the invention, there can be provided an intestinal environment-improving agent having an excellent intestinal environment-improving effect.

### BRIEF DESCRIPTION OF DRAWINGS

FIG. 1 is a graph showing the results of defecation frequencies in Examples of the invention and Comparative Examples.

FIG. 2 is a graph showing the results of stool outputs in Examples of the invention and Comparative Examples.

### EMBODIMENT FOR CARRYING OUT THE INVENTION

### Lactoferrin

As lactoferrin, mention is made of commercial lactoferrins, lactoferrins isolated, in a usual manner (e.g. ion exchange chromatography), from colostrum, transitional milk, normal milk, late lactation milk and the like of mammals (e.g. human beings, cow, sheep, goat, horse and the like), or treated products thereof such as defatted milk, whey and the like, lactoferrins produced from plants (such as tomato, rice plant, and tobacco), and lactoferrins obtained through genetic recombination. Lactoferrins may be commercially available ones or may be prepared by known techniques. These may be used singly or in combination of two or more. It will be noted that preferred lactoferrins are bovine-derived ones or enteric treated lactoferrins.

The amount of a lactoferrin relative to the total of the intestinal environment-improving agent is appropriately chosen depending on the type of preparation, administration form and administration subject and is preferably not smaller than 10 mg/day/adult and more preferably 50 to 5000 mg/day/adult. The effect of the invention can be obtained within this range. For instance, if 300 mg is taken in three tablets, one tablet should contain 100 mg, corresponding to ⅓ thereof.

[Bacteria of Lactobacillus brevis]

The bacterium is Lactobacillus brevis subsp. coagulans and is commonly called “Labre bacterium”. The bacteria have been found in a pickled vegetable called “Suguki-Zuke” and are well resistant to acids and salt, thus being resistant to gastric fluid and enteric fluid. They are delivered live to the intestines with strong power of remaining viable therein. The strains are not critical and may be used singly or in appropriate combination of two or more. Among them, it is preferred to use stain No. FERM BP-4693.

The amount of the bacteria of Lactobacillus brevis relative to the total of the intestinal environment-improving agent is appropriately chosen depending on the type of preparation, administration form and administration subject and is preferably not less than one hundred millions of viable bacteria, more preferably not less than one billion of viable bacteria and most preferably not less than ten billions of viable bacteria, each amount is amount per adult per day. As to killed bacteria, the amount is preferably not less than one billion, more preferably not less than ten billions and most preferably not less than eighteen billions. Both viable bacteria and killed bacteria are discharged when taken in large amounts and thus, their lower limit is not critical and generally not larger than ten trillions. Within theses ranges, a better effect of the invention can be obtained. It will be noted that Api 50CHL bioMerieux (made by bioMerieux Japan) is used for identification of lactobacilli. For the viable count of bacteria, anaerobic cultivation is performed by use of an MRS agar medium to measure the resulting grown colonies. With killed bacteria, the counted value, prior to sterilization treatment, obtained by use of the MRS agar medium like the viable bacteria was determined as the number of killed bacteria.

[Extract of a Plant Selected from Piperaceous, Zingiberaceous and Solanaceous Plants]

It is preferred to formulate, in the intestinal environment-improving agent of the invention, an extract of a plant selected from piperaceous, zingiberaceous and solanaceous plants so as to further improve the intestinal environment-improving effect.

More particularly, piperaceous plants include Piper nigrum L., Piper longum L., and Piper retrofractum Vahl, zingiberaceous plants include Zingiber officinale, and solanaceous plants include Capsicum annuum. These may be used singly or in combination of two or more.

As the above plant extract, there can be used commercially available products or those extracts obtained by known extract techniques. The solvents used for the extraction technique include: water; alcohols such as methanol, ethanol, propanol, butanol and the like; and polyhydric alcohols such as propylene glycol, butylene glycol and the like, which may be used singly or in the form of two or more mixed solvents thereof. Various conditions in the extraction method are not critical and a ratio between a starting material for extraction and an extraction solvent is preferably within a range of starting material for extraction: extraction solvent=about 1:2 to 1:50 on a weight ratio basis. The extraction temperature is preferably within a range of 5 to 80° C. and the extraction is preferably carried out for one hour to one week by immersing in an extraction solvent or under agitation. It will be noted that the extraction pH is not critical unless it is either extremely acidic or alkaline. If the extraction solvent consists of a non-toxic solvent such as water, ethanol, water/ethanol (hydrous ethanol) or the like, the resulting extract may be used as it is or in the form of a diluted solution. The extract may be provided as a concentrated extract, or may be prepared in the form of dried powder such as by freeze-drying or in the form of a paste. It is to be noted that if other type of solvent is used, it is preferred to use, after removal of the solvent by distillation, a dried matter diluted with a non-toxic solvent.

The amount of the plant extract selected from those of piperaceous, zingiberaceous and solanaceous plants relative to the total of the intestinal environment-improving agent is appropriately selected depending on the type of preparation, administration form and administration subject and is preferably at 0.1 to 5000 mg and more preferably 1 to 500 mg, per adult per day. Within this range, the effect of the invention can be obtained.

The intestinal environment-improving agent may further comprise, aside from the above ingredients, one or a combination of two or more arbitrary ingredients in appropriate amounts within ranges not impeding the effect of the invention. The arbitrary ingredients include, for example, oil ingredients, lubricants, excipients, disintegrants, binders, medicinal ingredients other than those set out hereinbefore, dyestuffs, essences and the like. More particularly, mention is made of the following ingredients.

Oil ingredients include various types of fatty acid esters, hydrocarbons, higher fatty acids, higher alcohols and the like. Lubricants include gum arabic, cacao oil, carnauba wax, hydrous silicon dioxide, dried aluminium hydroxide gel, glycerine, magnesium silicate, liquid paraffin, crystalline cellulose, sucrose fatty acid esters, stearyl alcohol, stearic acid, gelatin, lactose, saccharose, hydroxypropyl cellulose, hydroxypropyl methylcellulose, carboxymethylcellulose, fumaric acid, beeswax sugar and the like. Excipients include gum arabic, ethylcellulose, kaolin, cacao oil, fructose, silicon dioxide, xylitol, citric acid or salts thereof, crystalline cellulose, stearic acid or salts thereof, dextran, hydroxypropyl cellulose, hydroxypropyl methylcellulose, carboxymethylcellulose, polyvinylpyrrolidone, macrogol, calcium hydrogen phosphate, sodium hydrogen phosphate, sucrose, glucose, sorbitol, lactitol, corn starch, potato starch and the like. Disintegrants include cellulose or derivatives thereof, starch or derivatives thereof, and the like. Binders include hydroxypropyl cellulose, methylcellulose, carboxymethylcellulose, gelatin, vinylpyrrolidone, partially gelatinized starch and the like. Medicinal ingredients include carotenoid substances (α-carotene, β-carotene, 7-carotene, lycopene, lutein, astaxanthin, zeaxanthin and the like), coenzyme Q10, vitamin E, tocotorienol, DHA, EPA and the like.

The form of the intestinal environment-improving agent of the invention is not critical and includes liquid, powder, granule, tablet, capsule or the like. The intestinal environment-improving agent of the invention should preferably be an enteric coated agent. For the preparation of an enteric coated agent, it is convenient to formulate ingredients including shellac, water-soluble shellac, zein, hydroxymethyl cellulose phthalate, carboxymethylcellulose, cellulose acetate phthalate, methacrylic acid copolymers, ethyl cellulose, aminoalkylmethacylate copolymers, cell walls of beer yeast (e.g. a commercial name of Yeast Wrap or the like), tapioca starch, gelatin, pectin, fats and oils such as hardened oils, and the like. It will be noted that in the invention, whether an agent is an enteric coated agent or not is determined according to the disintegration testing method of the 14th revised Japanese Pharmacopoeia.

The method of preparing the intestinal environment-improving agent of the invention is not critical and is appropriately selected depending on the form of preparation. Mention is made of a method of preparing tablets wherein after mixing lactoferrin, bacteria of Lactobacillus brevis and arbitrary ingredients, the resulting mixture is subjected to compression molding. Further, it is preferred to carry out a method wherein using an enteric ingredient such as shellac, the tablets are coated to provide an enteric-coated preparation.

The method of ingesting the intestinal environment-improving agent differs depending on the form of preparation and is not critical and should preferably be administered along with water with the case of tablets. With other types of preparations, although the manner of ingestion is not critical, it is not favorable to heat them prior to ingestion because lactoferrin and viable bacterial of Lactobacillus brevis are not resistant to heat. The ingestion timing is not critical and ingestion should preferably be made from after dinner before bedtime.

In the invention, the intestinal environment improvement means to improve bowel and intestinal conditions, indicating that consistency of stool is kept proper and the defecation frequency and stool output are returned to normal ones. The intestinal environment-improving agent of the invention may serve as a quasi drug, a food for specified health use, a food or the like and may be conveniently applied as an antiflatulent, a laxative agent, an antidiarrheal, an aperient, an intestinal gas inhibitor or an abdominal fullness inhibitor. Moreover, as a consequence of the improved intestinal environment, there are obtained effects of improving cosmetic spread and skin's texture. Furthermore, the invention provides an intestinal environment-improving effective ingredients consisting of lactoferrin and bacteria of Lactobacillus brevis, an intestinal environment-improving effective ingredients lactoferrin, bacteria of Lactobacillus brevis and an extract of a plant selected from piperaceous, zingiberaceous and solanaceous plants, and an intestinal environment-improving method using these ingredients.

### EXAMPLES

Examples and Comparative Examples are shown to particularly illustrate the invention, which should not be construed as limited to the following Examples.

[Preparatory Examples of Stating Materials]
(1) Lactoferrin (MLF-EX, made by Morinaga Milk Industry Co., Ltd.)
(2) Viable bacteria of Lactobacillus brevis
   Isolated from a commercial beverage (plant-based lactobacillus Labre, made by Kagome Co., Ltd.) and anaerobically cultivated in an MRS medium (made by Oxoid Co., Ltd.), followed by harvest and freeze-drying. 60 mg of the resulting dried product corresponded to one hundred millions of viable bacteria. (Including strain No.: FERM BP-4693)
(3) Killed bacteria of Lactobacillus brevis
   Isolated from a commercial beverage (plant-based lactobacillus Labre, made by Kagome Co., Ltd.) and cultivated in an MRS medium, followed by harvest, sterilization at 121° C. for 20 minutes and freeze-drying. 36 mg of the resulting dried product corresponded to one billion of killed bacteria. (Including strain No.: FERM BP-4693)
(4) Long pepper extract (Hihatsu extract MF, made by Maruzen Pharmaceuticals Co., Ltd.)
(5) Excipient (mixture of lactose and crystalline cellulose in equal amounts)

### Examples 1 to 4, Comparative Examples 1 to 2

Ingredients other than shellac were mixed at such ratios as indicated in the following table and tableted into tablets having a diameter of 9 mm and a weight of 325 mg. Except for Example 4, the tablets were each coated with shellac (an enteric-coating agent) to provide enteric coated tablets. It will be noted that for identification of lactobacilli, Api 50CHL bioMerieux (made by bioMerieux Japan) was used. For the determination of viable count, anaerobic cultivation was performed using an MRS agar medium and grown colonies were counted. With killed bacteria, the value determined by use of an MRS agar medium, like viable bacteria, prior to sterilization treatment was taken as the number of killed bacteria. The resulting enteric coated tablets were evaluated in the following way. The results are also shown in the table.

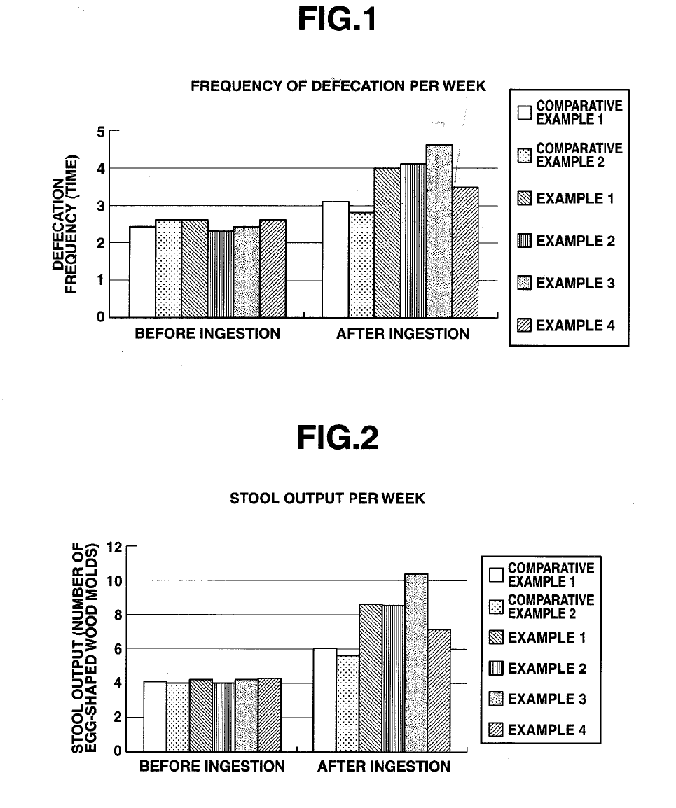

30 males and females, who tended to be constipated, were classified into six groups each consisting of five persons and were orally administered with three tablets per day from after dinner before bedtime over four weeks. The persons being tested recorded the defecation frequency and the stool output over one week prior to the administration and also over one week at the fourth week of the administration. The stool output was recorded, with respect to the stool size, in terms of the number of wood patterns while referring to an oval-shaped wood pattern (corresponding to an M size of Packed Egg Standards, regulated by the Ministry of Agriculture, Forestry and Fisheries of Japan). The average results of five persons are indicated in Table 1, the results of defecation frequency is shown in FIG. 1, and the results of the stool output is shown in FIG. 2.

TABLE 1

| | |
|---|---|
| Comparative | |
| Example | Example |

| (Amount mg/tablet) | | 1 | 2 | 1 | 2 | 3 | 4 |
|---|---|---|---|---|---|---|---|
| Lactoferrin | | 100 | — | 100 | 100 | 100 | 100 |
| Viable bacteria of | | — | 20 | 20 | — | 20 | 20 |
| Lactobacillus brevis | | | (hundred | (hundred | | (hundred | (hundred |
| (number of viable bacteria | | | millions) | millions) | | millions) | millions) |
| per three tables) | | | | | | | |
| Killed bacteria of | | — | — | — | 12 | — | — |
| Lactobacillus brevis | | | | | (one | | |
| (number of killed bacteria | | | | | billion) | | |
| per three tablets) | | | | | | | |
| Extract of long pepper | | — | — | — | — | 50 | — |
| Excipient | | balance | balance | balance | balance | balance | balance |
| Total of plain tablet (mg) | | 325 | 325 | 325 | 325 | 325 | 325 |
| Shellac | | 25 | 25 | 25 | 25 | 25 | — |
| Total of enteric coated | | 350 | 350 | 350 | 350 | 350 | 325 |
| tablet (mg) | | | | | | | |
| Defecation | Prior to | 2.4 | 2.6 | 2.6 | 2.3 | 2.4 | 2.6 |
| Frequency | ingestion | | | | | | |
| (times) | After | 3.1 | 2.8 | 4.0 | 4.1 | 4.6 | 3.5 |
| | ingestion | | | | | | |
| Stool output | Prior to | 4.1 | 4.0 | 4.2 | 4.0 | 4.2 | 4.3 |
| (number of | ingestion | | | | | | |
| pieces) | After | 6.1 | 5.7 | 8.7 | 8.6 | 10.5 | 7.2 |
| | ingestion | | | | | | |

Upon comparison with lactoferrin alone, combinations with lactoferrin and bacteria of Lactobacillus brevis and further with the long pepper extract become larger in the defecation frequency and stool output per week than prior to the ingestion, thereby showing an excellent intestinal environment-improving effect.

1. An intestinal environment-improving agent comprising a lactoferrin and bacteria of Lactobacillus brevis as effective ingredients.

2. The intestinal environment-improving agent as defined in claim 1, wherein said agent is an enteric coated agent.

3. The intestinal environment-improving agent as defined in claim 1 or 2, wherein said agent comprises not less than one hundred millions of viable bacteria of Lactobacillus brevis.

4. The intestinal environment-improving agent as defined in claim 1 or 2, wherein said agent comprises not less than one billion of killed bacteria of Lactobacillus brevis.

5. The intestinal environment-improving agent as defined in claim 1 further comprising an extract of a plant selected from piperaceous, zingiberaceous and solanaceous plants.
